(19) United States
(12) Patent Application Publication
(10) Pub. No.: US 2013/0165690 A1
(43) Pub. Date: Jun. 27, 2013

(54) METHOD FOR MANUFACTURING POLYMER-GRADE BIO-BASED ACRYLIC ACID FROM GLYCEROL

(71) Applicants: Michel Fauconet, Valmont (FR); Nabil Tlili, Mulhouse (FR)

(72) Inventors: Michel Fauconet, Valmont (FR); Nabil Tlili, Mulhouse (FR)

(73) Assignee: ARKEMA FRANCE, Colombes (FR)

(21) Appl. No.: 13/635,017

(22) PCT Filed: Mar. 15, 2011
(86) PCT No.: PCT/FR2011/050512

§ 371 Date: Oct. 22, 2012

(30) Foreign Application Priority Data
Mar. 18, 2010 (FR) 1051961

(51) Int. Cl. C07C 51/16

(52) U.S. Cl. CPC C07C 51/16
USPC 562/532; 562/598

(57) ABSTRACT

The present invention relates to a process for the manufacture of hioresourced acrylic acid of polymer grade having a content by weight of acrylic acid greater than 99% and the following contents of impurities: protoanemonin less than 5 ppm, total aldehydes less than 10 ppm, maleic anhydride less than 30 ppm, nonphenolic polymerization inhibitors less than 10 ppm, and a content by weight of ¹⁴C such that the ¹⁴C/¹²C ratio is greater than 0.8×10⁻¹².

The studies leading to the present invention benefited from financial support from the Seventh Framework Program of [The European Union] [The European Atomic Energy Community] ([FP7/2007-2013] [FP712007-2011]) under grant agreement No. 228867,

The present invention is targeted at a process for the manufacture of a bioresourced acrylic acid of polymer grade from glycerol as starting material. The term bioresourced indicates that the acrylic, acid is essentially based on a carbon source of natural origin.

Acrylic acid is a compound which is used as polymerization monomer or comonomer for the manufacture of a very broad range of final products. The polymers or copolymers are manufactured by polymerization of the acid or of the derivatives of this acid, in the ester (polyacrylates) or amide (polyacrylamides) form. These polymers are used as is or as copolymers in fields as varied as hygiene, detergents, paints, varnishes, adhesives, paper, textiles, leather, and the like. A very important outlet for acrylic acid is the manufacture of superabsorbents, in which a partially neutralized (mixture of acrylic acid and sodium acrylate or acrylates of other cations) acrylic acid is polymerized or else acrylic acid is polymerized and the polyacrylic compound obtained is partially neutralized.

Manufacturers have been developing processes for the synthesis of acrylic acid for decades.

The first generation used, as starting material, compounds comprising a triple bond of acetylenic type which were reacted with a mixture of carbon monoxide and water in the presence of a nickel-based catalyst,

The second generation of processes, which is today the most widely employed industrially, makes use of a reaction for the catalytic oxidation of propylene and/or propane using oxygen or an oxygen-comprising mixture.

This reaction is generally carried out in the gas phase and generally in two stages: the first stage carries out the substantially quantitative oxidation of the propylene to give an acrolein-rich mixture, in which acrylic acid is a minor component, and then the second stage carries out the selective oxidation of the acrolein to give acrylic acid.

The reaction conditions of these two stages, carried out in two reactors in series or in a single reactor comprising the two reaction stages in series, are different and require catalysts suited to the reaction; however, it is not necessary to isolate the acrolein from the first stage during this two-stage process.

The starting materials used result from oil or natural gas and consequently the acrylic acid is formed from a nonrenewable fossil carbon starting material, In addition, the processes for extracting, purifying and synthesizing the starting materials and the to processes for destroying, at the end of the cycle, the manunctured finished products based on these fossil starting materials generate carbon dioxide, the latter being a direct byproduct of the reactions of the oxidation of propylene to give acrolein and then of acrolein to give acrylic acid. All this contributes to increasing the concentration of greenhouse gases in the atmosphere. in the context of the commitments of the majority of industrialized countries to reduce emissions of greenhouse gases, it appears particularly important to manufacture novel products based on renewable starting materials, contributing to reducing these environmental effects.

For several years, manufacturers have been carrying out research and development studies on “bioresourced” synthetic processes using naturally renewable starting materials. Specifically, in order to limit the ecological impact of conventional production processes, alternative processes starting from nonfossil plant starting materials have recently been developed. Examples are processes using, as starting material, 2-hydroxypropionic acid (lactic acid) obtained by fermentation of glucose or molasses originating from the biomass. Further processes are those starting from glycerol (also known as glycerin), resulting from the methanolysis of vegetable oils at the same time as the methyl esters, which are themselves employed in particular as fuels in gas oil and domestic heating oil. The methanolysis of vegetable oils or animal fats can be carried out according to various well-known processes, in particular by using homogeneous catalysis, such as sodium hydroxide or sodium methoxide in solution in methanol, or by using heterogeneous catalysis. Reference may be made on this subject to the paper by D. Ballerini et al. in Pactuatite Chimique of November-December 2002.

The processes using hydroxypropionic acid as starting material have a major disadvantage from the economic viewpoint. They involve a fermentation reaction which is necessarily carried out under highly dilute conditions in water. In order to obtain acrylic acid, a very large amount of water has to be removed by distillation, at the price of a very high energy cost. Furthermore, the energy expended to separate the water, which energy is produced from fossil materials, will be highly damaging to the initial advantage of producing acrylic acid from this bioresourced starting material. Mention may be made, in this field, of application WO2006/092271, which describes a process for the production of polymers from acrylic acid prepared by the enzymatic route, in particular from carbohydrate.

It has been known for a long time that it is possible, starting from natural organic substances, such as polyols, capable of being converted by a chemical route, to obtain acids or aldehydes comprising 3 carbon atoms per molecule which can constitute precursors of acrylic acid. Mention may be made, by way of example, of the synthesis of acrolein obtained by dehydration of glycerol, which is described in particular in patent U.S. Pat. No. 5,387,720. Glycerol (also known as glycerin) results from the methanolysis of vegetable oils at the same time as the methyl esters, which are themselves employed in particular as fuels in gas oil and domestic heating oil. This is a natural product which enjoys a “green” aura, it is available in large amounts and it can be stored and transported without difficulty. Many studies have been devoted to giving economic value to glycerol according to its degree of purity and the dehydration of glycerol to give acrolein is one of the routes envisaged,

The reaction involved in order to obtain acrolein from glycerol is:

CH₂OH—CHOH—CH₂OH→CH₂═CH—CHO+2H₂O

This stage is followed by a stage of conventional oxidation of the acrolein in order to obtain the acrylic acid according to the reaction:

CH₂═CH—CHO+½O₂→CH₂═CH—COOH

Patent applications EP 1 710 227, WO2006/135336, WO2006/092272 and WO2007/0219521 describe processes for the synthesis of acrylic acid from glycerol comprising the stage of gas-phase dehydration in the presence of catalysts composed of inorganic oxides (which may or may not be mixed) based on aluminum, titanium, zirconium, vanadium, and the like, and the stage of gas-phase oxidation of the acrolein thus synthesized in the presence of catalysts based on oxides of iron, molybdenum, copper, and the like, alone or in combination in the form of mixed oxides.

However, they do not give precise details with regard to the phase of purification of the acrylic acid and do not describe at all what impurities are or are not present in the acrylic acid obtained. For example, application WO2006/092272 describes a process for the manufacture of acrylic acid and of superabsorbent from glycerol. It is asserted therein that it is possible to obtain an acrylic acid with a purity of 99 to 99.98% without specifying how it is obtained and what the residual impurities are. in example 2, it is simply specified that an acrylic acid which does not comprise protoanemonin is obtained without considering the other impurities capable of having an effect on the subsequent polymerizations.

Acrylic acid is intended for the use by manufacturers of processes for the polymerization either of acrylic acid or of its ester derivatives, which processes are carried out under various forms, in hulk, in solution, in suspension or in emulsion, These processes can be highly sensitive to the presence in the charge of certain impurities, such as aldehydes or unsaturated compounds, which can sometimes prevent the expected use value from being obtained, for example by limiting the conversion of the monomer to give the polymer, by limiting the chain length of the polymer or by interfering in the polymerization in the case of unsaturated compounds. Other impurities, such as nonpolymerizable saturated compounds, can be particularly troublesome in the final application by modifying the properties of the finished product, by conferring toxic or corrosive properties on the finished product or by increasing polluting organic discharges during the stages of manufacture of the polymer and/or of the finished product.

Operators are proving to be demanding as regards quality specifications for acrylic acid (or for its ester). The latter must meet strict thresholds as regards impurities. Specifically, users of acrylic acid or of acrylic esters which produce polymers employ formulations suited to the production of their polymers from a “standard” grade of acrylic acid or of esters today manufactured solely from propylene. A modification to the formulations used by these users, for the purpose of adapting them to a different grade of acrylic acid or of esters produced by a route other than that of the conventional processes starting from propylene, would exhibit significant disadvantages for these user companies. Apart from the additional research and development costs, the production of one type of polymer on the same unit starting from different grades of acrylic acid or of esters according to their origin, fossil or bioresourced (such as glycerol), would occasion significant conversion costs and a more complicated production infrastructure.

The need is now making itself felt for the marketing of an acrylic acid which meets all the abovementioned constraints, both upstream, that is to say an acrylic acid essentially based on a nonfossil natural carbon source, and downstream, that is to say an acrylic acid which meets quality standards allowing it to be used in the manufacture of a broad range of technical polymers, without, however, requiring a sophisticated and therefore expensive purification.

The use of carbon-based starting materials of natural and renewable origin can be detected by virtue of the carbon atoms participating in the composition of the final product. This is because, unlike substances resulting from fossil materials, substances composed of renewable starting materials comprise ¹⁴C. All carbon samples drawn from is living organisms (animals or plants) are in fact a mixture of 3 isotopes: ¹²C (representing ˜98.892%), ¹³C (˜1.108%) and ¹⁴C (traces: 1.2×10⁻¹⁰% ). The ¹⁴C/¹²C ratio of living tissues is identical to that of the atmosphere. In the environment, ¹⁴C exists in two predominant forms: in inorganic form, that is to say carbon dioxide gas (CO₂), and in organic form, that is to say carbon incorporated in organic molecules.

In a living organism, the ¹⁴C/¹²C ratio is kept constant metabolically as the carbon is continually exchanged with the environment. As the proportion of ¹⁴C is substantially constant in the atmosphere, it is the same in the organism, as long as it is living, since it absorbs the ¹⁴C like it absorbs the ¹²C. The mean ¹⁴C/¹²C ratio is equal to 1.2×10⁻¹².

¹²C is stable, that is to say that the number of ¹²C atoms in a given sample is constan over time. ¹⁴C for its part is radioactive and each gram of carbon of a living being comprises sufficient ¹⁴C isotope to give 13.6 disintegrations per minute.

The haiflife (or period) T_{1/2}, related to the disintegration constant of ¹⁴C, is 5730 years. Due to this period of time, the ¹⁴C content is regarded as constant in practice from the extraction of the plant starting materials to the manufacture of the final product.

The bioresourced acrylic acid of the invention has a content by weight of ¹⁴C such that the ¹⁴C/¹²C ratio is greater than 0.8×10⁻¹² and preferably greater than 1×10⁻¹². This bioresourced acrylic acid can even achieve a ratio equal to 1.2×10⁻¹² in the case where all of the carbon-based components used for its manufacture are of nonfossil or natural origin. Currently, there exist at least two different techniques for measuring the ¹⁴C content of a sample:
by liquid scintillation spectrometry
by mass spectrometry: the sample is reduced to graphite or to gaseous CO₂ and analyzed in a mass spectrometer. This technique uses an accelerator and a mass spectrometer to separate the ¹⁴C ions from the ¹²C ions and to thus determine the ratio of the two isotopes.

All these methods for measuring the ¹⁴C content of substances are clearly described in the standards ASTM D 6866 (in particular D6866-06) and in the standards ASTM D 7026 (in particular 7026-04). The measurement method preferably used is the mass spectrometry described in the standard ASTM D 6866-06 (accelerator mass spectroscopy).

The aim of the present invention is to overcome the previous disadvantages by providing a process for the manufacture of a bioresourced acrylic acid of polymer grade, this grade being defined by limiting thresholds for content of the impurities harmful to a broad range of polymerization processes.

The subject matter of the invention is a process for the manufacture of a bioresourced acrylic acid of polymer grade having a content by weight of acrylic acid >99% and the following contents of impurities:
total aldehydes <10 ppm
protoanemonin <5 ppm
maleic anhydride <30 ppm
nonphenolic polymerization inhibitors <10 ppm
and a content by weight of ¹⁴C such that the ¹⁴C/¹²C ratio>0.8×10⁻¹².

The acrylic acid obtained by the process of the invention will preferably have a content of
total aldehydes <3 ppm
protoanemonin <3 ppm
maleic anhydride <15 ppm
nonphenolic polymerization inhibitors <3 ppm and a content by weight of ¹⁴C such that the ¹⁴C/¹²C ratio >1×10⁻¹².

The invention is targeted at a process for the manufacture of an acrylic acid of polymer grade by using glycerol as starting material, which glycerol will be converted in two stages—dehydration and oxidation as mentioned above incorporated in an overall purification process.

This process is highly analogous to the synthetic process starting from propylene insofar as the intermediate product, acrolein, resulting from the first stage is the same and insofar as the second stage is carried out under the same operating conditions. However, the reaction of the first stage of the process of the invention, the dehydration reaction, is different from the reaction for the oxidation of propylene of the usual process. The dehydration reaction, performed in the as phase, is carried out using different solid catalysts from those used for the oxidation of propylene.

The acrolein-rich effluent resulting from the first dehydration stage, intended to feed the second stage of oxidation of acrolein to give acrylic acid, comprises a greater amount of water and in addition exhibits substantial differences as regards byproducts resulting from the reaction mechanisms involved given solid form by different selectivities in each of the two routes.

In order to illustrate these differences, the details relating to the presence of acetic acid (main impurity) in the crude acrylic acid, that is to say in the liquid phase exiting from the second-stage reactor, are collated in table 1 below.

TABLE 1

| Impurity/AA ratio by weight | Ex-propylene | Ex-glycerol |
|---|---|---|
| (crude acrylic acid) | process | process |
| Acetic acid/AA | <4% | >5% |

The impurities/AA ratios depend on the catalysts used, on their “age” (deterioration in the selectivities over time) and on the operating conditions.

Table 1 illustrates the main difference, in terms of constituents of the liquid effluent exiting from the oxidation reactor, between the ex-propylene and ex-glycerol processes. Naturally, although this is not mentioned in the table, a whole series of oxygen-comprising compounds, alcohols, aldehydes, ketones, some other acids in small amounts, and the like, are also found in the crude acrylic acid, whether it originates from the ex-propylene process or from the ex-glycerol process, the necessary separation of which is known to a person skilled in the art.

The specifications for the grades of acrylic acid currently used for the production of acrylic acid polymers and of acrylic esters require reducing the contents of impurities of table 1 in the acrylic acid down to the values which appear in table 2 below.

TABLE 2

| Concentration of the | | |
|---|---|---|
| impurities in the AA | Technical acrylic acid | Glacial acrylic acid |
| (by weight) | for esterification | for polymerization |
| Acetic acid | <0.2% | <0.1% |

Acetic acid is troublesome in particular because it is not converted during the polymerization process; it is saturated and thus nonpolymerizable. Depending on the polymerization process involved and the applications targeted for the polymer, this impurity may remain in the finished product and risk conferring undesirable corrosive properties on the finished product or it may he reencountered in the liquid or gaseous discharges generated by the polymerization process and may cause equally undesirable organic pollution.

The problem posed is that of obtaining an acrylic acid having a degree of purity corresponding to the requirements of the users and meeting the specifications: total aldehydes <10 ppm, protoanemonin <5 ppm, maleic anhydride <30 ppm, nonphenolic polymerization inhibitors <10 ppm, and also those given in table 2, by employing a process for the synthesis of acrylic acid using glycerol as starting material which exhibits the disadvantage, compared with the conventional process for the oxidation of propylene, of providing, at the outlet of the oxidation reactor, a gas mixture comprising a great deal of water and exhibiting high contents of acetic acid.

The contents of water and acetic acid in the effluent exiting from the oxidation reactor present a very great economic problem due to the expensive energy necessary for the removal of the water and the large number of treatments per distillation necessary for the separation of the acetic acid.

The applicant company has discovered that it is possible. to overcome the preceding disadvantages by employing a process for the purification of the gaseous effluent resulting from the oxidation reactor of a process for the synthesis of acrylic acid starting from glycerol, comprising a first stage of dehydration of the glycerol followed by a second stage of oxidation of the acrolein, combining a state of absorption of the acrylic acid by a heavy solvent at the oxidation reactor outlet and a multistage purification phase resulting in the acrylic acid of polymer grade.

Even if the state of the art describes various techniques which can be used in a process for the purification of acrylic acid, it cannot be established according to the prior art that the combination of these techniques would make it possible to obtain a bioresourced acrylic acid meeting the specifications of the users and overcoming the disadvantages inherent in the use of glycerol as starting material.

The use of a stage of absorption of the acrylic acid by a heavy solvent makes it possible to solve upstream the bulk of the problems presented by the presence of water and light impurities soluble in the aqueous phase. The phase of purification, by a is sequence of distillations, makes it possible to remove entirely simultaneously the traces of heavy impurities, of intermediate impurities, that is say those having a boiling point between that of the acrylic acid and that of the heavy solvent, essentially composed of “heavy” compounds originating from the dehydration and oxidation reactions, and possibly some polymerization-inhibiting stabilizing agents and light impurities which. remain.

The subject matter of the invention is a process for the manufacture of bioresourced acrylic acid of polymer grade having a content by weight of acrylic acid >99% and the following contents of impurities: total aldehydes <10 ppm, protoanemonin <5 ppm, maleic anhydride <30 ppm, nonphenolic polymerization inhibitors <10 ppm, and a content by weight of ¹³C such that the 14C/¹²C ratio >0.8×10⁻¹², from glycerol, which comprises the following stages:
i) dehydration of the glycerol to give acrolein,
ii) oxidation of the acrolein formed to give acrylic acid,
iii) extraction of the acrylic acid present in the effluent from the oxidation stage ii) by absorption in a column operating countercurrentwise by means of a hydrophobic heavy solvent, with cooling and removal at the top of the light fraction consisting of the “noncondensable” gaseous compounds and of the condensable light compounds, such as water, acetaldehyde, unconverted acrolein, formic acid and acetic acid,
iv) separation by topping by distillation of the liquid phase resulting from stage (iii) of a light fraction comprising water and residual light compounds, in particular acetic acid and formic acid, and of a heavy fraction comprising the acrylic acid in solution in the hydrophobic heavy solvent, the light fraction being recycled in the preceding extraction stage (iii),
v) distillation of the heavy fraction resulting from (iv) comprising the acrylic acid in solution with separation, at the bottom, of the hydrophobic heavy solvent and, at the top, of the acrylic acid fraction comprising the intermediate impurities and possibly traces of solvent, the heavy fraction resulting from this stage (v), essentially composed of the solvent, being recycled in stage (iii), optionally after a purification treatment,
vi) distillation of the acrylic acid solution from the top fraction resulting from the preceding stage (v) in order to extract, at the bottom, the heaviest “intermediate” compounds and the traces of solvent possibly entrained and, at the top, the technical acrylic acid,
vii) purification by distillation of the technical acrylic acid, in order to obtain an acrylic acid of “polymer” grade, after addition to the acrylic acid of an amino compound which reacts with the aldehydes still present.

In an alternative embodiment of the process, the gaseous reaction medium resulting from stage i) of dehydration of the glycerol, which has a high water content due to the glycerol charge (aqueous solution) and to the reaction itself, is subjected to an additional stage (i′) of partial condensation of the water. This additional stage is, for example, that described in patent application WO 08/087315 on behalf of the applicant; it will make it possible to eliminate a portion of the water, so as to bring this gas to a composition substantially identical to that of the ex-propylene process, in order to feed the second stage of oxidation of the acrolein to give acrylic acid. The water/acrolein molar ratio in the effluent resulting from the first stage of oxidation of the propylene is generally between 1,5/1 and 3/1, whereas it is of the order of 9/1 on conclusion of the stage of dehydration of the glycerol. Substantially identical composition is understood to mean in particular similar concentrations of acrolein, water and oxygen. This stage of partial condensation (i′) must be carried out with cooling to a temperature which makes it possible to obtain, after removal of the condensed phase, a gaseous stream comprising water and acrolein in a waterlacrolein molar ratio of 1.5/1 to 7/1. This partial condensation of the water, which requires lowering the temperature of the effluent exiting from stage i) at a temperature of between 280 and 320° C. in order to reach a temperature of 70 to 140° C., before increasing in order to achieve the conditions of the oxidation of the acrolein of stage ii), e.g. approximately 240° C., makes it possible, however, to avoid degradation of the catalyst of the 2nd stage of oxidation of the acrolein to give acrylic acid. It also makes it possible to avoid, during the subsequent stages, the expensive removal of large amounts of water, with the risk of resulting in losses of acrylic acid. In addition, it makes it possible to remove a portion of the “heavy” impurities formed during the dehydration.

Glycerol is a chemical, 1,2,3-proparietriol, which can be obtained either by chemical synthesis, starting with propylene, or as coproduct formed during the methanolysis of vegetable oils or animal fats. The methanolysis of vegetable oils, which constitutes a preliminary stage of the process in the case of integration of the entire oil/fat→acrylic acid line, results, on the one hand, in methyl esters and, on the other hand, in glycerol. The methyl esters are employed in particular as fuels in gas oil and domestic heating oil. With the development of fuels having renewable origins, in particular vegetable oil methyl esters (VOMEs), the production of glycerol according to this production route has greatly increased, the glycerol representing of the order of 10% of the weight of the oil converted.

The glycerin, the name of glycerol when it is in aqueous solution, obtained from vegetable oils or animal fats can comprise salts (NaCl, Na₂SO₄, KCl, K₂SO₄, and the like). In this case, a preliminary stage of removal of the salts, for example by distillation, by use of ion-exchange resins or by use of a fluidized bed, such as described in French application FR 2 913 974, will generally be present. Mention will in particular be made, among the methods used or studied for the purification and the evaporation of glycerol, of those which are described by G. B. D'Souza in J. Am. Oil Chemists' Soc., November 1979 (Vol 56) 812A, by Steinbemer U et al. in Fat. Sci, Technol. (1987), 89 Jahrgang No. 8, pp 297-303, and by Anderson D. D. et al, in Soaps and Detergents: A Theoretical and Practical Review, Miami Beach, Fla., Oct. 12-14 1994, chapter 6, pp 172-206. Ed: L Spitz, AOCS Press, Champaign.

Use is generally made of aqueous glycerol solutions having a concentration which can vary within wide limits, for example from 20 to 99% by weight of glycerol; preferably, use is made of solutions comprising from 30 to 80% by weight of glycerol.

The principle of the process for obtaining acrylic acid from glycerol is based on the 2 consecutive dehydration and oxidation reactions:

CH₂OH—CHOH—CH₂OH⇄CH₂═CH—CHO+2H₂O

CH₂═CH—CHO+½O₂→CH₂═CH—COOH

The process can be carried out in two separate stages with two different catalysts.

The dehydration reaction, which is an equilibrium reaction but one promoted by a high temperature level, is generally carried out in the gas phase in the reactor in the presence of a catalyst at a temperature ranging from 150° C. to 500° C., preferably between 250° C. and 350° C., and a pressure between 1 and 5 bar. It can also be carried out in the liquid phase. It can also be carried out in the presence of oxygen or of an oxygen-comprising gas, as described in applications WO 061087083 and WO 06/114506,

The oxidation reaction is carried out in the presence of molecular oxygen or of a mixture comprising molecular oxygen, at a temperature ranging from 200° C. to 350° C., preferably from 250° C. to 320° C., and under a pressure ranging from 1 to 5 bar, in the presence of an oxidation catalyst.

The glycerol dehydration reaction is generally carried out over solid acid catalysts. The catalysts which are suitable are homogeneous or multiphase substances which are insoluble in the reaction medium and which have a Hammett acidity, denoted H₀, of less than +2. As indicated in patent U.S. Pat. No. 5,387,720, which refers to the paper by K. Tanabe et al. in “Studies in Surface Science and Catalysis”, Vol. 51, 1989, chap. 1 and 2, the Hammett acidity is determined by amine titration using indicators or by adsorption of a base in the gas phase.

These catalysts can be chosen from natural or synthetic siliceous substances or acidic zeolites; inorganic supports, such as oxides, covered with mono-, tri- or polyacidic inorganic acids; oxides or mixed oxides or heteropolyacids or heteropolyacid salts.

These catalysts can generally be composed of a heteropolyacid salt in which the protons of said heteropolyacid are exchanged with at least one cation chosen from elements belonging to Groups I to XVI of the Periodic Table of the Elements, these heteropolyacid salts comprising at least one element chosen from the group consisting of W, Mo and V.

Mention may particularly be made, among mixed oxides, of those based on iron and on phosphorus and of those based on cesium, phosphorus and tungsten.

The catalysts are chosen in particular from zeolites, Nation® composites (based on sulfonic acid of fluoropolymers), chlorinated aluminas, phosphotungstic and/or silicotungstic acids and acid salts, and various solids of the type comprising metal oxides, such as tantalum oxide Ta₂O₅, niobium oxide Nb₂O₅, alumina Al₂O₃, titanium oxide TiO₂, zirconia ZrO₂, tin oxide SnO₂, silica SiO₂ or silicoaluminate SiO₂/Al₂O₃, impregnated with acid functional groups, such as borate BO₃, sulfate SO₄, tungstate WO₃, phosphate PO₄, silicate SiO₂ or molybdate MoO₃′ functional groups, or a mixture of these compounds.

The preceding catalysts can additionally comprise a promoter, such as Au, Ag, Cu, Pt, Rh, Pd, Ru, Sm, Ce, Yt, Sc, La, Zn, Mg, Fe, Co, Ni or montmorillonite.

The preferred catalysts are phosphated zirconias, tungstated zirconias, silica zirconias, titanium or tin oxides impregnated with tungstate or phosphotungstate, phosphated aluminas or silicas, heteropolyacids or heteropolyacid salts, iron phosphates and iron phosphates comprising a promoter.

Use is made, as oxidating catalyst, of any type of catalyst well known to a person skilled in the art for this reaction. Use is generally made of solids comprising at least one element chosen from the list Mo, V, W, Re, Cr, Mn, Fe, Co, Ni, Cu, Zn, Sn, Te, Sb, Bi, Pt, Pd, Ru and Rh, present in the metallic form or in the oxide, sulfate or phosphate form. Use is made in particular of the formulations comprising Mo and/or V and/or W and/or Cu and/or Sb and/or Fe as main constituents.

When the process is carried out in two stages with two different reactors, it can be advantageous to carry out, between the two reactors, an intermediate condensation of the water, as described in application WO 08/087315.

The conversion of the glycerol to give acrylic acid, which is based on the 2 abovementioned consecutive dehydration and oxidation reactions, can also be carried out in one and the same reactor. In this reaction scheme, known as oxydehydration, the exothermic nature of the oxidation reaction is compensated for by the endothermic nature of the dehydration reaction, which contributes to a better thermal balance of the process. In processes of this type, it is possible either to use two different catalytic beds, an upstream dehydration bed and a downstream oxidation bed, each with their specific catalyst, or a single bed with a “mixed” catalyst composed of a mixture of the dehydration and oxidation catalysts. This version of the process comprising a single reactor involves a recycling (after separation) of the unconverted acrolein in order to carry out the oxidation phase.

The gas mixture resulting from the 2^{nd} stage (oxidation reaction) is composed, apart from acrylic acid:
of light compounds which are rioncmdensable under the temperature and pressure conditions normally employed: nitrogen, unconverted oxygen, carbon monoxide and carbon dioxide, which are formed in a small amount by final oxidation,
of condensable light compounds: in particular water, generated by the dehydration reaction or present as diluent, unconverted acrolein, light aldehydes, such as formaldehyde and acetaldehyde, formic acid and acetic acid,
of heavy compounds: furfuraldehyde, benzaldehyde, maleic acid, maleic t ydride, benzoic acid, phenol, protoanemonin, and the like.

The second stage of the manufacture consists in recovering the acrylic acid present in the gaseous effluent resulting from the oxidation reaction in order to convert it into acrylic acid of polymer grade of the invention.

The first stage of this purification stage (stage iii of the process according to the invention) consists of an extraction of the acrylic acid by countercurrentwise absorption which is accompanied by a cooling of the whole. For this, the gaseous effluent resulting from the reactor is introduced at the bottom of an absorption column where it encounters, countercurrentwise, a hydrophobic heavy solvent or a mixture of hydrophobic heavy solvents introduced at the column top. The flow rate of solvent introduced at the column top is from 3 to 6 times by weight that of the acrylic acid present in the gaseous effluent for feeding the absorption column. The light compounds, under the temperature and pressure conditions normally employed (respectively more than 50° C. and less than 2×10⁵ Pa), are removed at the top of this absorption column.

A heavy solvent solution having a content of acrylic acid generally of between 15 and 25% by weight and additionally comprising “intermediate” compounds, having a boiling point between that of the heavy solvent and that of the acrylic acid, is collected at the column bottom. These intermediate compounds consist of heavy reaction products: furfuraldehyde, benzaldehyde, maleic acid, maleic anhydride, benzoic acid, phenol and protoanemonin, and possibly of certain stabilizing products introduced into the medium in order to inhibit the polymerization reactions.

The light fraction, exiting at the top, consists of light compounds which are noncondensable under the temperature and pressure conditions normally employed: nitrogen, unconverted oxygen, carbon monoxide and carbon dioxide, which are formed in a small amount by final oxidation, and of condensable light compounds: in particular water, generated by the dehydration reaction or present as diluent, unconverted acrolein, light aldehydes, such as formaldehyde and acetaldehyde, formic acid and acetic acid.

This operation of extraction by a hydrophobic heavy solvent is well known and has even been described for the treatment of acrylic acid synthesized by oxidation of propylene. Mention may be made, on this subject, of the following patents: French patent No. 1 588 432, French patent No. 2 146 386, German patent No. 4 308 087, European patent No. 0 706 986 and French patent No. 2 756 280, which describe such solvents, These solvents have a boiling point of greater than 170° C., for example of between 200 and 380° C. and preferably between 270 and 320° C. French patent No, 1 588 432 describes the use of aliphatic or aromatic acid esters having a high boiling point. They generally consist of binary mixtures capable of forming eutectics, such as, for example, biphenyl (BP) and diphenyl ether (DPO), which form a eutectic in a proportion of 26.5-76.5 (FP No. 2 146 386 and EP 0 706 986), or even a ternary mixture, BP/DPO/dimethyl phthalate (DMP) (DE No. 4 308 078). French patent No. 2 756 280 recommends the use of aromatic solvents exhibiting a boiling point of greater than 260° C. and comprising one or two aromatic nuclei substituted by at least one alkyl radical having from 1 to 4 carbon atoms or one cycloalkyl radical, in particular ditolyl ether, alone or in the form of a mixture of its isomers, or the ditolyl ether (DTE) and dimethyl phthalate mixture.

The process of the invention can be carried out with these different solvents. However, the preferred solvents are those described in this French patent No. 2 756 280, which, apart from the fact that they improve the separation of the impurities present in the reaction mixture, make possible the efficient recovery of the polymerization inhibitors. The operating conditions for this absorption stage are as follows: The gaseous reaction mixture is introduced at the column bottom at a temperature of between 130° C. and 250° C. The hydrophobic heavy solvent is introduced at the column top at a temperature of between 10° C. and 60° C. The respective amounts of solvent and of gaseous reaction mixture are such that the heavy solvent/acrylic acid ratio by weight is between 3/1 and 6/1. The operation is carried out at a pressure close to atmospheric pressure of between 0.8 and 2×10⁵ Pa.

In the second purification stage, stage (iv) of the process of the invention, the liquid solution of acrylic acid in the hydrophobic heavy solvent is subsequently sent to a topping region, in order to remove, at the top, the traces of water and of condensable light compounds which have remained at the bottom of the preceding absorption region. This topping region is fed at the top with the bottom stream from the absorption region. The stream extracted at the top, enriched in light compounds, is returned to the absorption region of stage iii), for the purpose of removing these liEht compounds in the top stream from this absorption region and of collecting, at the bottom, the acrylic acid in solution in the hydrophobic heavy solvent.

The third stage of this purification phase (stage v) is a distillation of the heavy fraction resulting from stage (iv), comprising the acrylic acid, in order to separate, at the bottom, the heavy solvent and, at the top, an acrylic acid fraction comprising the intermediate impurities and possibly traces of solvent, the heavy fraction, essentially consisting of the hydrophobic heavy solvent, resulting from this stage (v) being recycled to stage (iii), optionally after a purification treatment.

The fourth stage of this purification phase (stage vi) is a stage of separation by distillation, on the one hand, of the intermediate compounds and, on the other hand, of the purified acrylic acid (technical acrylic acid), The bottom stream from the preceding column of stage (v) is introduced at the bottom of a distillation column operating under a top pressure of the order of 2×10³ to 2×10⁴ Pa. A stream of purified acrylic acid, of technical grade, is obtained at the top and the intermediate compounds are obtained at the bottom. The technical acrylic acid produced is subsequently sent, during a stage vii), to a final purification region which makes it possible to achieve the acrylic acid of polymer grade. The fifth stage of this purification phase (stage vii) consists in finally purifying the acrylic acid of technical grade to give acrylic acid of polymer grade. In order to achieve this grade of acrylic acid, which makes it possible to synthesize polymers of high molecular weight, it is particularly important to remove certain residual aldehydes, such as furfuraldehyde, benzaldehyde and acrolein, down to extremely low contents, which cannot be achieved economically by a simple distillation due to their volatility, which is too close to that of acrylic acid. In order to do this, the aldehydes can be removed by a chemical treatment using a reactant which forms, with these aldehydes, heavy reaction products which can be more easily separated from the acrylic acid by distillation. Use may be made, among the reactants which can be employed, of amines, as described in patent U.S. Pat. No. 3,725,208, and more particularly of the compounds of the family of the hydrazines, such as glycine, as described in patent JP 7,500.014, or hydrazine hydrate, as described in patents U.S. Pat. No. 3,725,208 or JP 7,430,312, or aminoguanidine, as described in patent EP 270 999. These compounds can be used as is or in the form of their salts. The chemical treatments which are described all exhibit the is disadvantage of generating water during the reaction of the aldehyde with the amino reactant. The presence of water in the acrylic acid can also be harmful to the manufacture of certain polymers. For this reason, it can be advantageous to carry out this chemical treatment operation together with a distillation stage targeted at first removing the water and the light compounds at the top, before a stage of distillation of the acrylic acid intended to separate the heavy compounds, as is described in patent JP 7,495,920.

In a preferred embodiment of the process of the invention, the stream of technical acrylic acid is conveyed as feed of a distillation column operating under a top pressure of the order of 2×10³ to 2×10⁴ Pa, as a mixture with the amino reactant for removal of the aldehydes chosen from hydrazine derivatives, preferably hydrazine hydrate, introduced in a molar ratio of 2 to 10 with respect to the aldehydes present in the technical acrylic acid. The column top stream, composed essentially of acrylic acid, of water and of acetic acid, the latter two in a low concentration, can be recycled upstream of the process, preferably to the extraction stage iii, in order to recover the acrylic acid. The column bottom stream is, for its part, conveyed to the bottom of a second column operating under a top pressure of the order of 2×10³ to 2×10⁴ Pa, in which the heavy compounds are removed at the bottom and the acrylic acid is distilled at the top, in order to obtain a “polymer wrade” of acrylic acid.

The various stages of separation by absorption or distillation require the addition of polymerization inhibitors to the treated streams, in order Co prevent the formation of heavy polymeric compounds prejudicial to the satisfactory operation of the assembly. The polymerization inhibitors generally used for the stages for the purification of acrylic acid are phenolic products, such as hydroquinone or hydroquinone methyl ether, phenothiazine derivatives, compounds of the family of the thiocarbamates, such as copper di(n-butyl)dithiocarbarnate, amino derivatives, such as hydroxylamines, hydroxydiphenylamine or derivatives of the family of the phenylenediamines, nitroxide derivatives of 2,2,6,6-tetramethylpiperidine-1-oxyl (TEMPO), such as 4-hydroxy-TEMPO or 4-oxo-TEMPO, or metal salts, such as manganese acetate. These inhibitors can be used alone or in combination and are in addition preferably introduced in combination with an oxygen-comprising gas.

These polymerization inhibitors are generally heavy compounds, the volatility of is which is lower than that of acrylic acid, but can, in some cases, be lighter than the solvent. They are removed at the bottom of the columns, when heavier inhibitors than the solvent are concerned, or are distributed between the top stream and the bottom stream, for inhibitors lighter than or similar to the solvent, In the majority of the columns, their concentration in the vapor phase inside the distillation columns is low and insufficient to prevent the initiation of polymers. In order to prevent the appearance and the accumulation of polymers, these additives are usually introduced into the liquid streams feeding the devices, but also at the top and at various points of the columns and devices, an as to provide continuous and homogeneous reflux of solution rich in polymerization inhibitors over all the parts of the devices. Generally, they are conveyed in solution in a liquid, for example in the acrylic acid or in the solvent, if the purification stage relates to streams comprising the solvent.

The acrylic acid of polymer grade obtained at the top of the final distillation column (stage vii) is additivated with hydroquinone methyl ether (HQME), at a concentration of 200+/−20 ppm; the final product is stabilized by addition of this inhibitor to the column top stream.

The invention also relates to the use of bioresourced acrylic acid of polymer grade in the manufacture of superabsorbents, comprising the polymerization of said partially neutralized acid or the polymerization of said acid, followed by a partial neutralization of the polyacrylic acid obtained.

The invention also relates to the superabsorbents obtained by polymerization of bio-acrylic acid of polymer grade.

The invention is also targeted at the use of bioresourced acrylic acid of polymer grade in the manufacture of polymers or of copolymers by polymerization of the derivatives of said acid in the ester or amide form. It is also targeted at the polymers or copolymers obtained by polymerization of the derivatives, in the ester or amide form, of bioresourced acrylic acid of polymer grade.

A better understanding of the process of the invention will be obtained on reading the examples below, given simply by way of illustration.

### EXAMPLES

The acrylic acid of the invention and its process of manufacture are illustrated in the following examples:
Example 1: preliminary stage of purification of the crude glycerol.
Example 2: conversion of the glycerol to acrolein, followed by partial condensation of the water.
Example 3: oxidation of acrolein to give acrylic acid.
Example 4: recovery of the crude acrylic acid in the form of a solution in the heavy solvent.
Example 5: separation of the solvent/crude acrylic acid in order to obtain technical acrylic acid.
Example 6: purification of the technical acrylic acid in order to obtain polymer grade acrylic acid.

In the examples which follow, the very low concentrations of impurities in the technical acrylic acid and polymer grade acrylic acid streams are measured by the following methods (figures in brackets: accuracy; quantification threshold):
by high performance liquid chromatography on a Lichrospher 100-RP-18 column, with detection by UV spectrometry and quantification by external calibration: protoanemonin (3%; 0.1 ppm), furfural (1.4%; 0.1 ppm), benzaldehyde (0.2%; 0.25 ppm), maleic anhydride assayed in the form of maleic acid (1.5%; 0.1 ppm); phenothiazine (2%; 0.2 ppm);
by the same method, with preliminary derivatization in the presence of dinitrophenylhydrazine: formaldehyde (3%; 0.1 ppm);
by UV/visible spectrometry, after reaction of acrolein with 4-hexylresorcinol in an ethanatrichloroacetic acid medium catalyzed by mercuric chloride and development of a blue coloration exhibiting a maximum absorbance at 603 nm: acrolein (5%; 0.1 ppm);
by gas chromatography on an FRAP column, with detection by flame ionization and quantification by internal calibration: acetic acid (3%; 10 ppm).

### Example 1

The first phase consists in purifying the crude glycerol obtained from the methanolysis of vegetable oils, with removal of the salts. The crude glycerol solution comprises, by weight, 88.5% of glycerol, 5.1% of water and 5.1% of sodium chloride. A stream of 8642 g is continuously conveyed as feed over a 2 liter stirred reactor heated by an external electrical reactor heater. The glycerol and water vapors are condensed in a reflux condenser and recovered in a receiver. This purification operation is carried out under a pressure of 670 Pa. 7695 g of a glycerol solution devoid of sodium chloride are obtained.

### Example 2

In a second phase, the reaction for the dehydration of the glycerol to give acrolein is carried out. The dehydration reaction is carried out in the gas phase in a fixed bed reactor in the presence of a solid catalyst composed of a tungstated zirconia ZrO₂/WO₃ at a temperature of 320° C. at atmospheric pressure. A mixture of glycerol (50% by weight) and water (50% by weight) is conveyed to an evaporator in the presence of air in an O₂/glycerol molar ratio of 0,6/1, The gas medium exiting from the evaporator at 290° C. is introduced into the reactor, composed of a tube with a diameter of 30 mm charged with 400 ml of catalyst and immersed in a salt bath (KNO₃, NaNO₃ and NaNO₂ eutectic mixture) maintained at a temperature of 320° C.

In order to carry out the optional stage of condensation of a portion of the water, the gaseous reaction mixture is conveyed, at the outlet of the reactor, to the bottom of a condenser. This condenser is composed of a column comprising a ProPak packing, equipped with a water-cooled top condenser. The cooling temperature in this exchanger is adjusted so as to obtain, at the gas outlet, a temperature of the vapors of 64° C. at atmospheric pressure. Under these conditions, the loss of acrolein at the condensation column bottom is less than 2%,

### Example 3

In a third phase, the gas mixture comprising 1.75 mall of acrolein is introduced, after addition of air (O₂/acrolein molar ratio of 0.9/1) and of nitrogen in an amount necessary in order to obtain an acrolein concentration of 5.4 mol %, as feed of the reactor for the oxidation of acrolein to give acrylic acid. This oxidation reactor is composed of a tube with a diameter of 30 mm charged with 480 ml of catalyst based on Mo/V mixed oxide and immersed in a salt bath, identical to that described in example 2, maintained at a temperature of 250° C. Before introducing over the catalytic bed, the gas mixture is preheated in a tube which is also immersed in the salt bath.

The composition of the reaction gas mixture at the reaction outlet (% by volume) is as follows: nitrogen (66%), oxygen (2%), water (15%), CO₂ (7%), CO (1%), acrylic acid (5.7%) and acetic acid (0.5%).

### Example 4

At the outlet of the oxidation reactor, the gas mixture is introduced at the bottom of an absorption column operating under absolute pressure of 130 kPa in order to carry out the stage (iii). This column comprises 8 theoretical plates in total, in the lower part, over ¼ of its total height, the column is equipped with a condensation section, at the top of which is recycled a portion of the condensed mixture recovered at the column bottom, after cooling to 67° C. in an external exchanger. A stream consisting of DTE (ditolyl ether) and DMP (dimethyl phthalate) in a proportion of a DTE/DMP ratio by weight of 0.85/0.15, with a solvent/acrylic acid present in the reaction gas ratio by weight of 5/1, in which 1% of HOME and 0.01% of copper dibutyldithiocarbamate have been dissolved beforehand as polymerization inhibitors, is fed at the column top at a temperature of 50° C. The temperature of the column top vapors is 65° C. and that of the acrylic acid solution obtained at the column bottom is 83° C. The product obtained at the bottom is cooled to a temperature of 64° C. and is then sent, using a pump, to the top of a second column with an efficiency of 9 theoretical plates. The distillation is carried out in this column under a pressure of 107 hPa The column bottom temperature is 97° C. and the column top temperature is 72° C. All the vapors condensed at the top are conveyed to the external cooling loop of the absorption column.

### Example 5

The stream extracted at the bottom of the second column of example 4 is a solution of crude acrylic, acid in the hydrophobic heavy solvent which assays approximately 17% of io acrylic acid. The stream of crude acrylic acid solution obtained in the preceding stage is cooled to 45° C. and then conveyed as feed to the top of a distillation column with an efficiency of 3 theoretical plates, said column operating under a pressure of 67 hPa. At the column top, a portion of the extracted and condensed stream is returned at the level of the upper plate and the other portion is recovered. The temperature in the boiler is 152° C. and the top temperature reaches 82° C.

The AA stream withdrawn at the column top, which comprises 98.2% of acrylic acid with, as major impurities, 0.17% of acetic acid, 0.33% of maleic anhydride and 0.06% of ditolyl ether, is sent to the bottom part of a second column of 11 theoretical plates, operating under a pressure of 140 hPa, and receives, at the top, a stabilizing agent mixture (5% FIQME in AA). The reflux ratio applied at the top (flow rate of liquid refluxed/flow rate of liquid withdrawn) is 1.5/1. The bottom temperature is 95° C. and the top temperature is 85° C.

The technical acrylic acid obtained at the top of the latter column assays 99.6% of AA. The impurities present in this stream are acetic acid (0.17%), furfural (0.005%), protoanemonin (0.009%), benzaldehyde (0.012%), maleic anhydride (0.05%) and water (0.1%),

### Example 6

The acrylic acid of technical grade resulting from example 5 is additivated with hydrazine hydrate in a molar ratio of 7/1 with respect to the aldehydes present (furfural, benzaldehyde, acrolein, and the like) and the stream is distilled in a column of 10 theoretical plates operating under a pressure of 90 hPa, at the top of which is extracted 10% of the feed stream, with a reflux ratio/withdrawal of 1/1. The stream obtained at the bottom of this column is subsequently sent as feed to a final column of 10 theoretical plates, under a pressure of 90 hPa, with a top temperature of 69° C.

The analyses of the polymer grade acrylic acid obtained at the top of this column show that the product comprises I ppm of protoanemonin, 0.8 ppm of furfural, 0.5 ppm of benzaldehyde, 0.2 ppm of acrolein, <1 ppm of formaldehyde, 0.08% of acetic acid and 12 ppm of maleic anhydride.

The acrylic acid obtained, analyzed by the ASTM D 6866-08 method, exhibits a content by weight of ¹⁴C corresponding to a proportion of bioresourced product/fossil product of greater than 99%.

1. A process for the manufacture of hioresourced acrylic acid of polymer grade, from glycerol, said polymer grade comprising by weight, acrylic acid in an amount greater than 99% and protoanemonin in an amount less than 5 ppm, aldehydes in an amount less than 10 ppm, maleic anhydride in an amount less than 30 ppm, nonphenolic polymerization inhibitors in an amount less than 10 ppm, and a content by weight of ¹⁴C such that the ¹⁴C/¹²C ratio is greater than 0.8×10⁻¹², the process comprising the following stages:
i) dehydration of the glycerol to produce acrolein,
ii) oxidation of the acrolein to produce acrylic acid,
iii) extraction of the acrylic acid present in effluent from the oxidation stage ii) by absorption. in a column operating countercurrentwise using hydrophobic heavy solvent,
iv) separation by topping a distillation of a liquid phase resulting from stage (iii) of a light fraction comprising water and residual light compounds, and of a heavy fraction comprising acrylic acid in solution in the hydrophobic heavy solvent,
v) distillation, of the heavy fraction resulting from (iv) comprising the acrylic acid in solution with separation, at the bottom, of the hydrophobic heavy solvent and, at the top, of the acrylic acid fraction comprising intermediate impurities and optionally traces of solvent, the heavy fraction resulting from this stage (v), essentially composed of the solvent, being recycled in stage (iii), optionally after a purification treatment,
vi) distillation of acrylic acid solution from the top fraction resulting from the preceding stage (v) in order to extract, at the bottom, intermediate compounds and solvent and, at the top, technical acrylic acid,
vii) purification by distillation of the technical acrylic acid, in order to obtain polymer grade acrylic acid, after addition to the acrylic acid of an amino compound present.

2. The process as claimed in claim 1, characterized in that stage iii) employs at least one water-immiscible absorption solvent having a boiling point of greater than 170° C.

3. The process as claimed in claim 2, characterized in that the absorption solvent consists of a solvent selected from the group consisting of ditolyl ether, a mixture of its ditolyl ether isomers, and a mixture of ditolyl ether and dimethyl phthalate.

4. The process as claimed in claim 1, characterized in that the gaseous reaction mixture resulting from stage ii) is introduced at the column bottom at a temperature of between 130° C. and 250° C., the hydrophobic heavy solvent being introduced at the column top at a temperature of between 10° C. and 60° C., and the respective amounts of solvent and of gaseous reaction mixture are such that the heavy solvent/acrylic acid ratio by weight is between 3/1 and 6/1, the operation being carried out at a pressure close to atmospheric pressure of between 0.8 and 2×10⁵ Pa.

5. The process as claimed in claim 1, characterized in that stages i) and ii) are carried out in a single reaction region.

6. The process as claimed in claim 1, characterized in that stages and ii) are carried out in two successive reaction regions.

7. The process as claimed in claim 6, characterized in that a partial condensation of the water is carried out between stages and ii) at. a temperature of between 70° C. and 140° C.

8. The process as claimed in claim 1, characterized in that a heavy solvent solution having a content of acrylic acid of between 15 and 25% by weight and further comprising intermediate compounds, having a boiling point between that of the heavy solvent and that of the acrylic acid, consisting of the heavy reaction products, is collected at the bottom of the absorption column of stage iii), which solution is then sent to a region for topping by distillation, stage iv), in order to remove, at the top, water and condensable compounds which have remained at the bottom of the absorption region of stage iii) and to collect, at the bottom, acrylic acid in the hydrophobic heavy solvent.

9. The process as claimed in claim 1, characterized in that, during stage v), the heavy fraction resulting from stage (iv), comprising the acrylic acid, is distilled in order to separate, at the bottom, the heavy solvent and, at the top, an acrylic acid fraction comprising the intermediate impurities and solvent, the heavy fraction, essentially consisting of the hydrophobic heavy solvent, resulting from this stage (v) being recycled to stage (iii), optionally after a purification treatment, and then, during stage vi), intermediate compounds, and technical acrylic acid are separated by distillation.

10. The process as claimed in claim 1, characterized in that stage vi) is carried out in a column operating under a pressure of between 2×10³ and 2×10⁴ Pa.

11. The process as claimed in claim 1, characterized in that the acrylic acid resulting from stage vi) is subjected to a further distillation carried out in the presence of compounds capable of reacting with the aldehydes said compounds chosen from the group consisting of amines, hydrazines, including their salts.

12. Acrylic acid obtained according to the process of claim 1.
